(19) United States
(12) Patent Application Publication
(10) Pub. No.: US 2016/0095735 A1
(43) Pub. Date: Apr. 7, 2016

(54) ANKLE-FOOT ORTHOTIC FOR TREATMENT OF FOOT DROP

(71) Applicant: JE3.LLC, St. Paul, MN (US)

(72) Inventor: Jerry Wenger, Powell, WY (US)

(21) Appl. No.: 14/874,707

(22) Filed: Oct. 5, 2015

Related U.S. Application Data
(60) Provisional application No. 62/059,600, filed on Oct. 3, 2014.

(51) Int. Cl. A61F 5/01

(52) U.S. Cl. CPC A61F 5/0127

(57) ABSTRACT

A customizable ankle-foot orthotic for treating foot drop and steppage gate that that enables a wearer to adjust the fit of the orthotic against the wearer's ankle for the purpose of reducing pressure points while ensuring adequate support. The orthotic includes an ankle brace which has a first wing defining a first and second tab which are independently configurable to overlap and selectively couple to the second wing enabling the wearer to customize the fit of the interior face against the wearer's ankle.

### RELATED APPLICATION

The present application claims the benefit of U.S. Provisional Application No. 62/059,600 filed Oct. 3, 2014, which is hereby incorporated herein in its entirety by reference.

### FIELD OF THE DISCLOSURE

The invention relates generally to treating steppage gait in persons suffering from a foot drop condition, and more specifically, to an apparatus and related methods for supporting the front portion of the foot to prevent foot drop and alleviate the steppage gait caused by the foot drop.

### BACKGROUND OF THE DISCLOSURE

Foot drop, also commonly known as drop foot, is a common condition in which a person is unable to lift the front portion of one or both feet during walking or running due to a neurological or muscular condition which weakens the muscles of the foot. The condition causes the front portion of the person's foot to slap down on the floor or drag across the floor during walking or running. Persons suffering from foot drop often compensate by lifting the afflicted foot higher than normally necessary when moving to prevent the dropping foot from dragging or slapping on the floor. The irregular gait caused by the foot drop and the compensating movement by the person is known as a steppage gait.

Foot drop is typically treated by ankle-foot orthotics that either lock the person's ankle in place or support the front portion of the person's foot. However, the currently available ankle-foot orthotics often restrict the natural flexing motion of the ankle and foot during normal movement to prevent the foot drop motion, replacing the steppage gait with a different irregular gait. Ankle locking orthotics, such as the one described in U.S. Pat. No. 3,916,886, herein incorporated by reference, typically comprise rigid L-shaped members fitted against the back of the ankle and the underside of the foot to prevent the entire foot from flexing downwardly at the ankle. However, because the L-shape member completely prevents downward flexing of the ankle at the foot, the foot cannot make the slight downward flexing movement that naturally occurs during walking or running. Ankle-foot orthotics which provide support to the front portion of the foot also impede the natural flexing of the foot and ankle during a natural gait. These orthotics typically comprise straps anchored to the person's calf or ankle at one end and anchored to the person's foot or shoe at the other end as disclosed in U.S. Pat. Nos. 4,817,589 and 7,458,950, both of which are herein incorporated by reference, to provide a tensile force preventing downward motion of the foot. However, the straps used are often static or only have a limited elasticity to ensure sufficient tensile force is applied to support the front portion of the foot. The inelasticity of the straps also prevents the natural flexing of the foot and ankle during normal movement. The limited flexibility of presently available ankle-foot orthotics effectively replace the irregular steppage gait with an irregular gait.

In addition to being overly restrictive, currently available ankle-foot orthotics are often too bulky to fit easily into shoes. Similarly, currently available ankle-foot orthotics often require numerous straps or anchors to properly secure the orthotic to the person's ankle or foot increasing the difficulty of putting the orthotic on or taking it off. For example, the L-shaped members of ankle locking orthotics are often large and do not easily fit into shoes without extensive modification of the shoe. In addition, the rigidness of the L-shape member prevents wearers from pointing their toes to ease putting on or taking off of the shoe. Similarly, strap orthotics often employ a plurality of straps and anchors that must be attached to the shoe or foot to properly fasten the orthotic to the person's ankle and foot. The straps and the anchors may be difficult to remove from the shoe if the person wants to remove or change shoes. The complexity and the difficulty of putting on and taking off the currently available orthotics often cause wearers to spend considerable time putting on or taking off the orthotic or forgoing the orthotic altogether.

An additional problem for strap orthotics is the positioning of the straps and locating where the straps are to be anchored on the wearer. Typically, the anchor locations for the straps are placed as high up on the calf as possible and as far down the foot or shoe as possible to achieve the maximum tensile force. However, the higher up the calf or further down the foot the anchor points are placed, the more visible and cumbersome the orthotic becomes for the user. Similarly, the greater the number of straps, the more support is provided for the foot. As with the positioning of the anchor locations, the greater the number of straps, the more visible and cumbersome the orthotic becomes for the wearer. Similarly, the positioning of the straps may be uncomfortable for users if the straps are pressed against the person's foot or ankle. The straps may rub against the person's legs or feet during movement causing chaffing or other discomfort.

### SUMMARY OF THE DISCLOSURE

An embodiment of the present invention is directed to an ankle-foot orthotic for applying a pulling force to a wearer's shoe to counteract the effects of drop foot and prevent steppage gait. More specifically, the ankle-foot orthotic anchors the top of the wearer's shoe to an ankle brace around the wearer's ankle with an elastic strap. The elastic strap applies a tensile force to the top of the wearer's shoe to prevent steppage gait while still providing the necessary flexibility to enable the wearer to flex their foot and ankle naturally during walking or running. The ankle-foot orthotic also comprises a highly customizable engagement system for linking the orthotic to the wearer's shoe allowing the wearer to customize where the tensile force is directed and the amount of tensile force applied, while at the same time allowing the wearer's foot or shoe to flex normally when walking.

An ankle-foot orthotic for treating steppage gait according to an embodiment of the present invention generally comprises an ankle brace and an elastic strap. The ankle brace further comprises wings for wrapping around a wearer's ankle and also defines an exterior face and an interior face contacting the wearer's ankle. In an embodiment of the present invention, an ankle pad can be positioned between the wearer's ankle and interior face of the ankle brace to prevent discomfort to the wearer while wearing the ankle-foot orthotic. A primary strap loop for receiving the elastic strap is positioned on the exterior face of the ankle brace between the first and second wings. In operation, wrapping the ankle brace around the wearer's ankle positions the primary strap loop at the rear of the wearer's ankle.

According to an embodiment of the present invention, the ankle-foot orthotic includes an ankle brace, a tension assembly, and an elastic strap. The ankle brace is adapted for installation around a wearer's ankle. The ankle brace can include an exterior face and an interior face, wherein the interior face is positionable against the wearer's ankle. The ankle brace can further include a first wing and a second wing, wherein the first wing defines a first tab and a second tab that are independently configurable to overlap and selectively couple to a portion of the second wing to enable the wearer to customize the fit of the interior face against the wearer's ankle. The tension adjustment assembly can be positioned on the exterior face of the ankle brace so as to protrude outwardly from the rear of the ankle brace when the ankle brace is wrapped around the wearer's ankle. The tension adjustment assembly can include one or more strap loops. The elastic strap can operably link the ankle brace to the wearer's shoe for applying an upward resistive force to the wearer's shoe to prevent inadvertent downward movement of a wearer's foot. The elastic strap can be threadable through the one or more strap loops such that the elastic strap wraps around the exterior face of the ankle brace and extends at a downward angle to engage the wearer's shoe.

The elastic strap defines a first end and a second end and is adapted to engage the wearer's shoe. The elastic strap can further comprise a hook fastener disposed at each end for engaging any looped structures on the wearer's shoes such as shoe laces or eyelets for receiving shoe laces. The hook fasteners enable for easy engagement of the shoe and customization as to the exact location that the tensile force provided by the elastic strap will be applied. Alternatively, the elastic strap can further comprise a strap adjustment mechanism having a first buckle and a second buckle positioned on the elastic strap. In this configuration, the first and second ends of the elastic strap are threaded through the looped structure on the shoe and looped back to the first and second buckle respectively. The strap adjustment mechanism enables the wearer to change the effective length of the elastic strap and correspondingly the tension applied by the elastic strap to the wearer's shoe.

According to an embodiment of the present invention, in operation, the elastic strap is threaded through the primary strap loop and wrapped around the wearer's ankle to engage the wearer's shoe. In this arrangement, the elastic strap can be angled at an ideal angle to provide the correct tension force for maintaining the wearer's shoe in the correct position. Alternatively, the ankle brace can be wrapped around the wearer's ankle such that the primary strap loop is disposed at the front of the wearer's angle. In this configuration, the elastic strap is not wrapped around the wearer's ankle and instead directly engages the wearer's shoe.

According to an embodiment of the present invention, the ankle-foot orthotic can further comprise ring anchors engageable to any looped structure on the wearer's shoe, including the shoe lace eyelets. The ring anchors can be easily engaged by or disengaged from the hook fasteners of the elastic strap. Consequently, the ring anchors can significantly reduce the time necessary to put on and take off the ankle-foot orthotic and the customization in that wearers can quickly try out a plurality of positions and arrangements for engaging the wearer's shoe with the elastic strap.

According to an embodiment of the invention, the orthotic can further comprise a support fitted to the exterior of the ankle brace. The support is adapted to protect the rear of the user's ankle from chaffing or other discomfort from the elastic strap rubbing against the user's ankle during movement. The ankle support can comprise a rigid polymer to provide support for the user's ankle and to prevent the user from feeling any discomfort from the shifting or stretching of the elastic strap while walking. The ankle support is countered to protect the user's ankle without inhibiting the user's flexibility so as to enable the user to walk without hindrance.

According to an embodiment of the present invention, the support can further comprise a strap adjustment assembly having an adjustable primary strap loop and two secondary strap loops positioned within cutouts in the support. The elastic strap can be threaded through the primary and secondary strap loops to form a U-shaped portion in the elastic strap. The length of the adjustable primary strap loop can be adjusted to increase or decrease the size of the U-shaped portion, which correspondingly changes the effective length of the elastic strap and the tension applied to the wearer's shoe. Alternatively, the strap adjustment assembly can comprise a fixed length primary strap loop and two secondary strap loops. In this configuration, the support comprises a plurality of cutouts for receiving the primary strap loop. Positioning the primary strap loop in the various cutouts changes the size of the U-shaped portion and correspondingly the tension applied to the wearer's shoe.

According to an embodiment of the present invention, a fastener clip can comprise an anchor portion, a first longitudinal side portion, a curved end portion, a second longitudinal side portion, and an end portion. The fastener hook can be inserted and removed from an eyelet and can have a structure capable of anchoring the fastener clip in a proper position while enabling rotation and movement of the fastener clip. A hook fastener can engage with the curved end portion to provide tension force.

According to an embodiment, an ankle brace can be shaped to conform to the ankle area and have a built-in conformable support fixedly sandwiched between the interior face and the exterior face of the brace. The built-in conformable support can be comprised of a plurality of longitudinal support members of varying lengths and configurations and can be comprised of a stiff yet flexible material. The length of each individual longitudinal support member is chosen so that the ankle-foot orthotic can be form fitting and provide sufficient structure and support while allowing mobility of the ankle and reducing pressure points. A strap loop is fixedly attached to the built-in conformable support.

The above summary of the invention is not intended to describe each illustrated embodiment or every implementation of the present disclosure. The Figures and the Detailed Description that follow more particularly exemplify these embodiments.

### BRIEF DESCRIPTION THE FIGURES

The disclosure can be more completely understood in consideration of the following detailed description of various embodiments of the disclosure, in connection with the accompanying drawings, in which:

FIG. 1 is a perspective view of an ankle-foot orthotic in an unrolled configuration presenting the exterior face of the orthotic in accordance with an embodiment of the disclosure.

FIG. 2 is a perspective view of the ankle-foot orthotic of FIG. 1 in an unrolled configuration presenting the interior face.

FIG. 3 is an exploded view of the ankle-foot orthotic of FIG. 1.

FIG. 4 is cross sectional view of the ankle-foot orthotic of FIG. 1 in an unrolled configuration.

FIG. 5 is a side view of an ankle-foot orthotic in accordance with an embodiment of the disclosure.

FIGS. 6-10 are perspective views of an ankle-foot orthotic in accordance with an embodiment of the disclosure being attached to a user's ankle and foot.

FIG. 11 is a perspective view of a fastener clip in accordance with an embodiment of the disclosure.

FIGS. 12-13 are perspective views of the fastener clip of FIG. 11 in an operational position.

FIG. 14 is a side view of a grommet assembly in accordance with an embodiment of the disclosure.

FIG. 15 is a perspective view of a double hooked heel holder in accordance with an embodiment of the disclosure.

FIG. 16 is a perspective view of a loop heel holder in accordance with an embodiment of the disclosure.

FIG. 17 is a perspective view of a hookless heel holder in accordance with an embodiment of the disclosure.

FIG. 18 is a perspective view of an ankle-foot orthotic with an integrated multi-member support in accordance with an embodiment of the disclosure.

FIG. 19 is a perspective view of an ankle-foot orthotic with an integrated single-member support in accordance with an embodiment of the disclosure.

FIG. 20 is a perspective view of the ankle-foot orthotic of FIG. 20 showing the exterior face in accordance with an embodiment of the disclosure.

FIGS. 21-25 are perspective views of an ankle-foot orthotic with an integrated single-member support in accordance with an embodiment of the disclosure being attached to a user's ankle and foot.

While embodiments of the disclosure are amenable to various modifications and alternative forms, specifics thereof have by shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure as defined by the appended claims.

### DETAILED DESCRIPTION

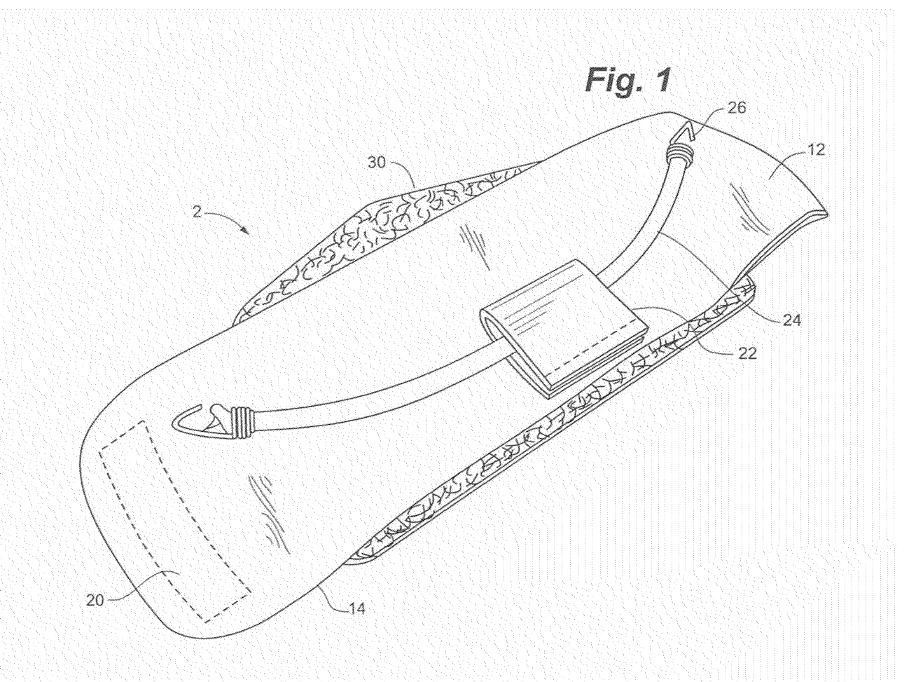

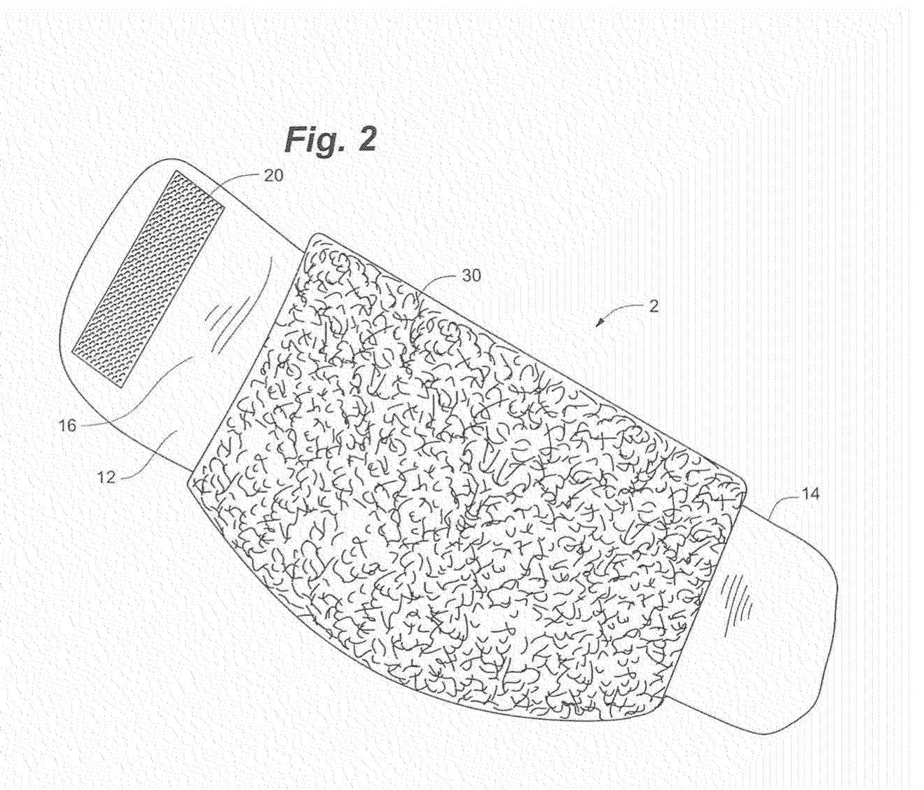

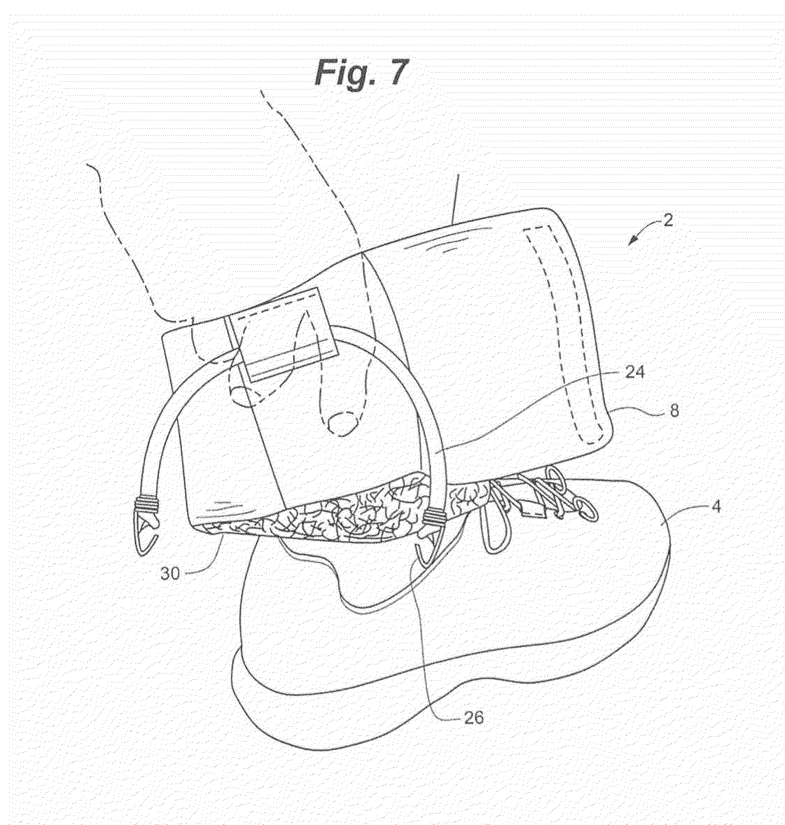

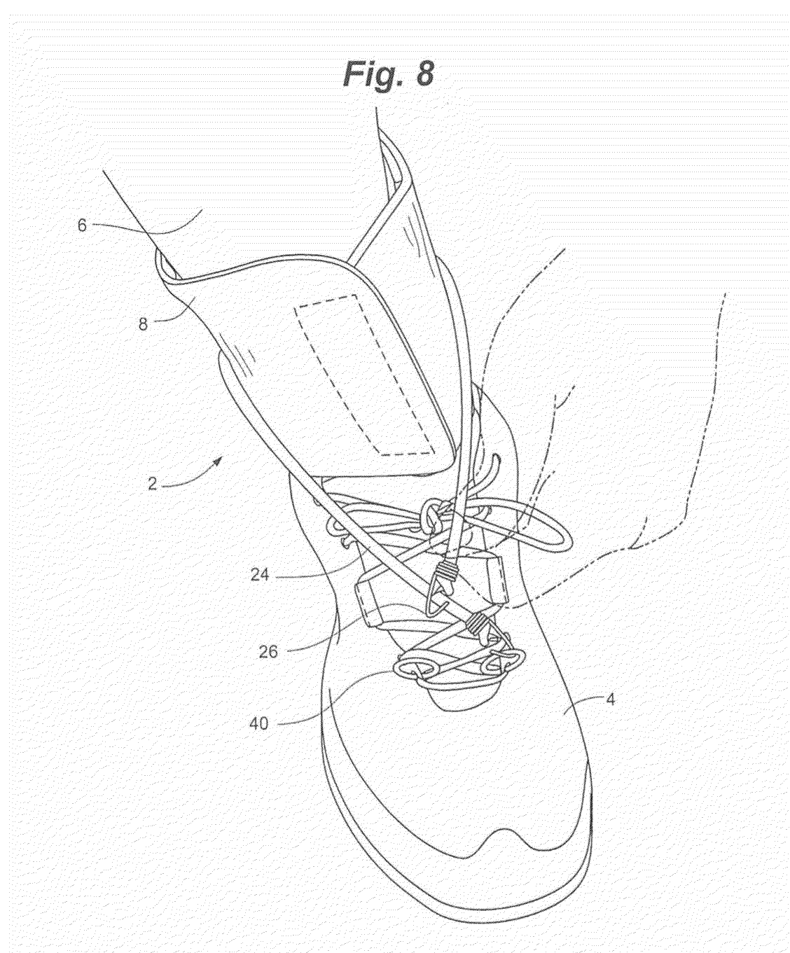

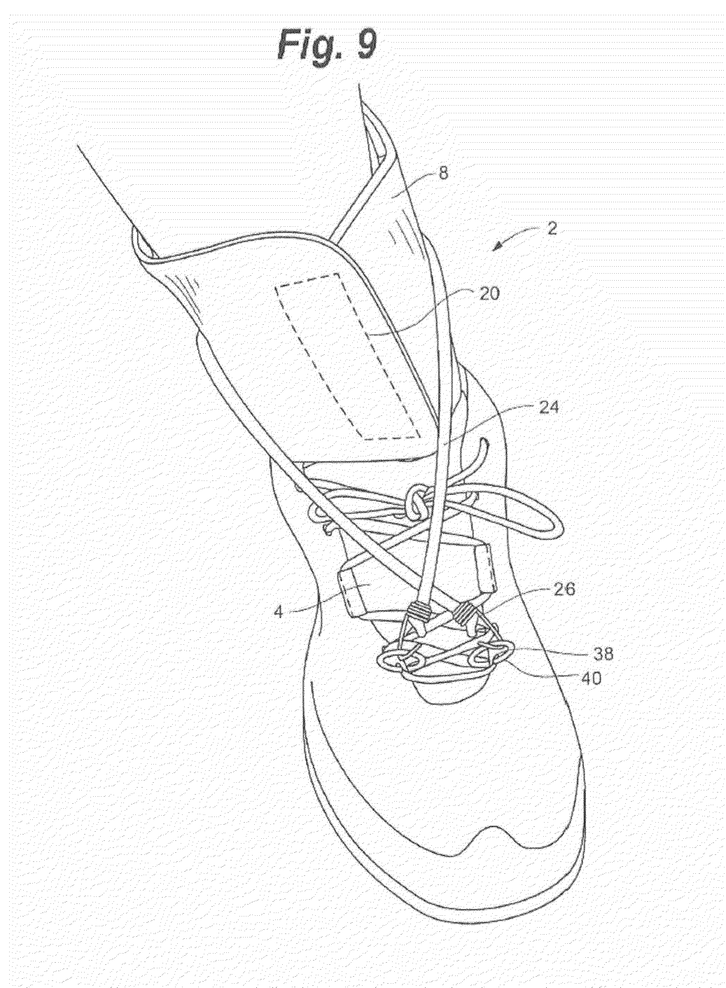

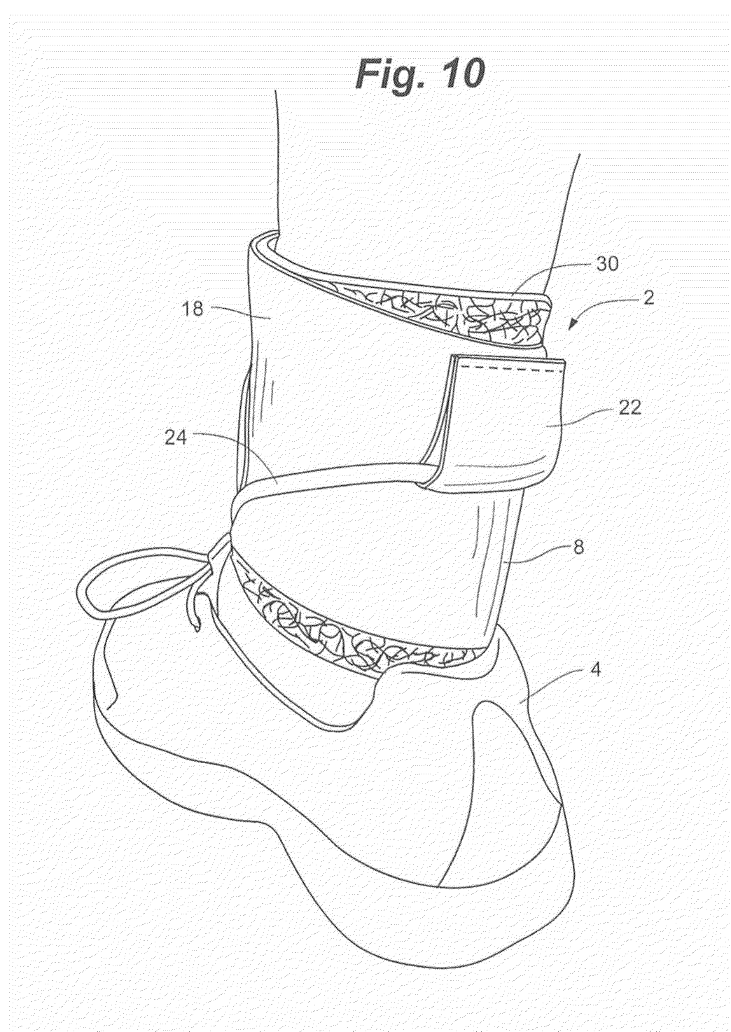

Referring to FIGS. 1-10, an ankle-foot orthotic 2 for treating steppage gait according to an embodiment of the present invention supports the weakened muscles by providing support beneath the wearer's foot or shoe 4 or by applying a pulling force to top of the wearer's shoe 4, while at the same time allowing the wearer's foot or shoe 4 to flex normally when walking. The pulling force is typically provided by anchoring the front or top of wearer's foot or shoe 4 to the wearer's ankle 6.

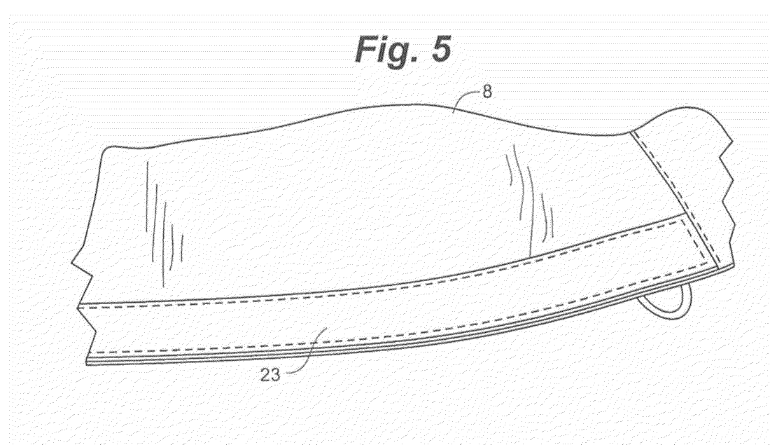

With special references to FIGS. 1-5, in one embodiment, ankle-foot orthotic 2 generally comprises an ankle brace 8 and an elastic strap 24. In one embodiment, ankle brace 8 can comprise a first wing 12 and a second wing 14 and defines an interior face 16 and an exterior face 18. Ankle brace 8 can further comprise an ankle brace fastener 20 for releasably engaging the first wing 12 to the second wing 14 such that the ankle brace 8 forms a generally cylindrical shape around a wearer's ankle 6. Ankle brace fastener 20 can comprise, for example, VELCRO®, a hook and loop fastener, an elastic fastener, a button fastener. Similarly, ankle brace 8 can be elastic material adapted to pull against the wearer's ankle 6 to maintain the position of the ankle brace 8 on the wearer's ankle 6. A primary strap loop 22 is disposed on the exterior face 18 of the ankle brace 8 between the first and second wing 12, 14. In one embodiment, ankle brace 8 can further comprise a structural strap 23 (shown in FIG. 5) for maintaining the shape of ankle brace 8 during movement and as tension force is applied to ankle brace 8. Structural strap 23 can comprise a flexible plastic material sufficiently rigid to withstand the tension forces applied to the ankle brace 8, but also sufficiently flexible to wrap around the user's ankle 6.

Elastic strap 24 defines a first end 27 and second end 28. Elastic strap 24 is threadable through the primary strap loop 22 and comprises an elastic material for applying a pulling force to the wearer's shoe 4. In one embodiment, different elastic straps 24 can be interchanged according the particular pull force required to prevent steppage gait or provide the desired flexibility for the wearer. Elastic strap 24 is sufficiently elastic to provide sufficient tensile force to prevent the foot from inadvertently dropping due to foot drop, but has sufficient flexibility to enable the wearer to flex their foot and ankle 6 as needed. As such, a wearer can walk or run using the ankle-foot orthotic 2 with the natural flexing of the foot and ankle 6.

In one embodiment, elastic strap 24 can comprise a hook fastener 26 disposed at both the first and second ends 27, 28. Hook fasteners 26 are adapted to engage any looped structure on the wearer's shoes 4 to operably link the ankle brace 8 to the wearer's shoes 4. In one embodiment, the hook fasteners 26 can be shaped such that the point of the hook is substantially parallel to the shank of the hook or points away from the shank to facilitate easy attachment and removal of the hook from the looped structures on the top of the shoe 4.

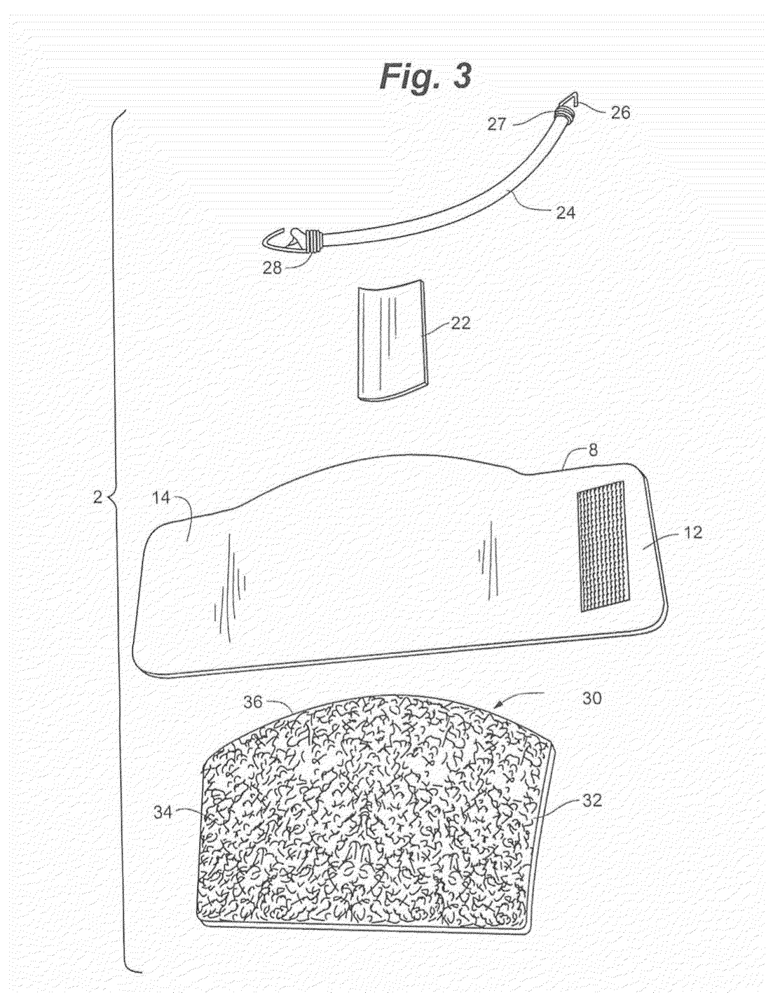

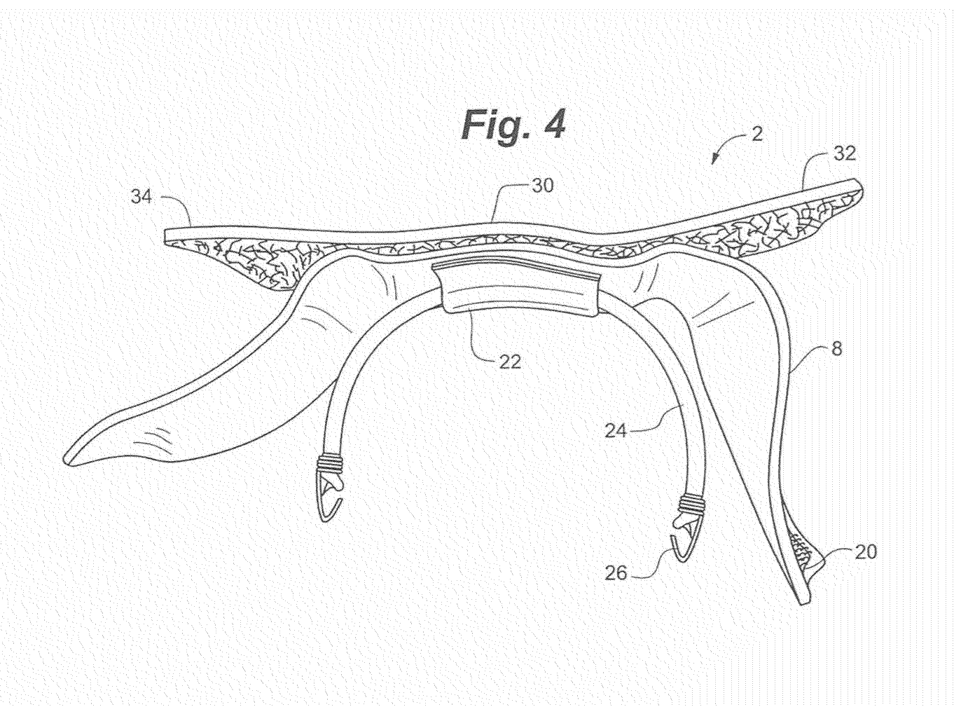

In one embodiment, ankle brace 8 can further comprise an ankle pad 30 for protecting the wearer's ankle 6 from discomfort. Ankle pad 30 can be disposed on the interior face 16 of the ankle brace 8 such that the ankle pad 30 is positioned between the ankle brace 8 and the wearer's ankle 6 when orthotic 2 is in use. Ankle pad 30 can comprise a foam, a mesh or other cushioning material to alleviate wearer discomfort. In one embodiment, the ankle pad 30 can be positioned on the ankle brace 8 such that the ankle pad 30 is positioned against the rear of the wearer's ankle 6 when the ankle brace 8 is wrapped around the wearer's ankle 6. In this configuration, the ankle pad 30 prevents the elastic strap 24 from chaffing or causing other discomfort to the wearer's ankle 6 when the elastic strap 24 is wrapped around the wearer's ankle 6 to engage the wearer's shoe 4. Alternatively, ankle pad 30 can further comprise a first wing 32 and a second wing 34 (shown in FIG. 4) adapted to wrap around the wearer's ankle 6 to protect both the sides and rear of the wearer's ankle 6 from discomfort. According to an embodiment of the present invention, the ankle pad 30 can further comprise a shoe cushion 36 (shown in FIG. 3) for preventing chaffing between the wearer's shoe 4 and the ankle pad 30. The shoe cushion 36 similarly supports the ankle pad 30 to prevent the ankle brace 8 from sliding down the wearer's ankle 6 during use.

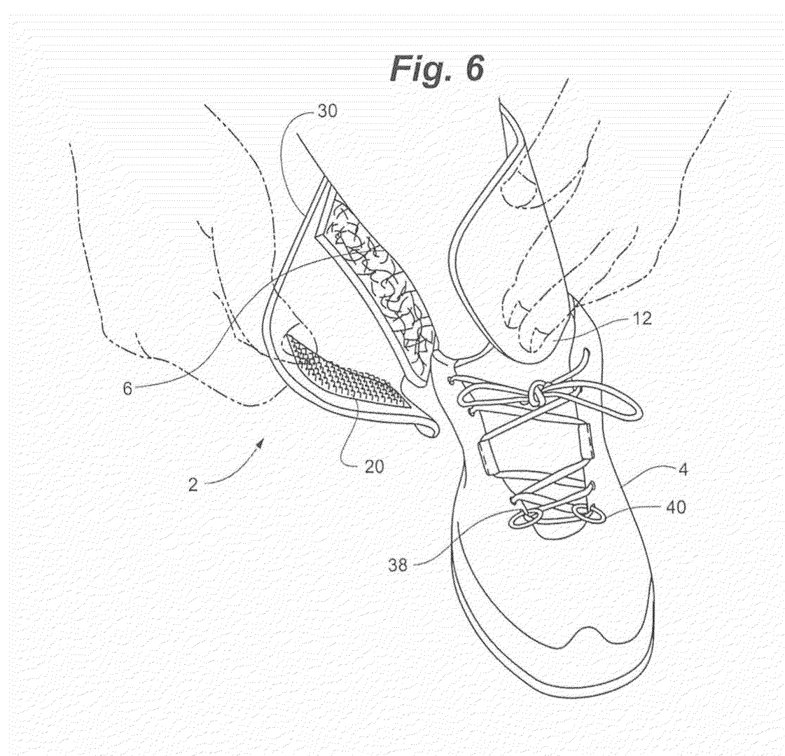

Referring to FIGS. 6-10, in operation, the first and second wings 12, 14 of the ankle brace 8 are wrapped around the wearer's ankle 6 such that primary strap loop 22 extends outwardly from the rear of the wearer's ankle 6. In this configuration, elastic strap 24 is wrapped around ankle brace 8 and the wearer's ankle 6 before engaging the wearer's shoes 4. Wrapping the elastic strap 24 around the wearer's ankle 6 ensures the correct tension is applied along elastic strap 24 and properly angles the elastic strap 24 to maintain the wearer's shoe 4 in the proper position. Alternatively, the wings 12, 14 of ankle brace 8 can be wrapped around the wearer's ankle 6 such that the primary strap loop 22 is positioned at the front of the wearer's ankle 6.

Elastic strap 24 comprises hook fasteners 26 for engaging any looped structure on the wearer's shoe 4. Typical shoes 4 can comprise eyelets 38 for receiving shoes laces. Hook fasteners 26 can engage either the laces, eyelets 38 or any other looped structure commonly found on conventional shoes 4. According to one embodiment (as depicted in FIGS. 6-10), at least one ring anchor 40, threaded through at least one of the eyelets 38, is employed to engage hook fasteners 26. In some embodiments, ring anchor 40 can be moved to whichever eyelet 38 best suits the needs of the wearer.

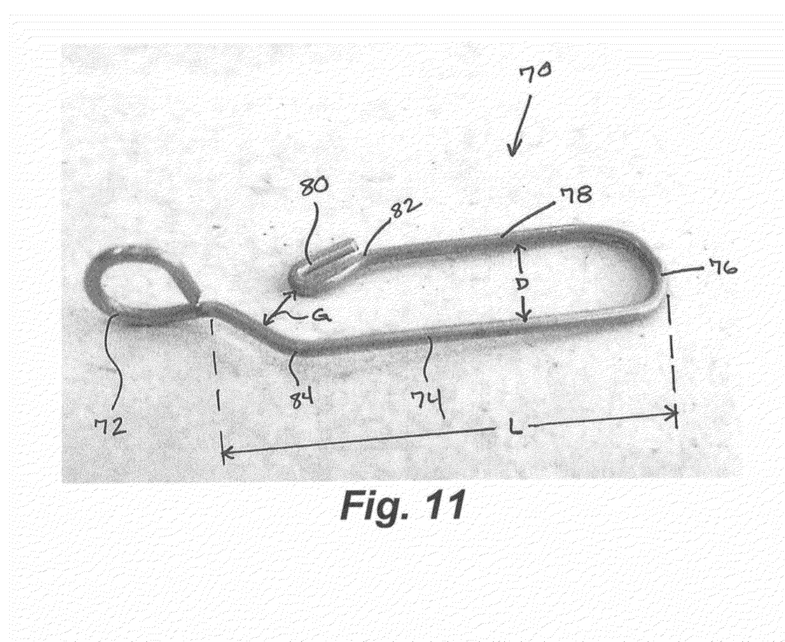

In another embodiment (as depicted in FIGS. 11-13), a fastener clip 90 can be threaded through at least one of the eyelets 38 and can be employed to engage hook fasteners 26. In one embodiment, fastener clip 90 is configured to be easily inserted and removed from eyelet 38 and has a structure configured to anchor fastener clip 90 in a proper position while enabling rotation and movement of fastener clip 90. In one embodiment, fastener clip 90 can comprise an anchor portion 92, a first longitudinal side portion 94, a curved end portion 96, a second longitudinal side portion 98, and an end portion 91.

Anchor portion 92 can be configured to provide a dimension or area larger than that of eyelet 38 so that anchor portion 92 cannot be threaded through eyelet 38. First longitudinal side portion 94 and second longitudinal side portion 98 can be substantially parallel to each other and in one embodiment are connected via curved end portion 96. In one embodiment, first longitudinal portion 94 can be substantially straight. In another embodiment, first longitudinal portion 94 can be configured to have at least one bend 99. First longitudinal portion 94 has a length L where the length L can be determined based on the configuration of the wearer's shoe.

First longitudinal side portion 94 and second longitudinal side portion 98 are a distance D from each other that is sufficient for the material of the wearer's shoe to be encompassed within. End portion 91 connects to second longitudinal portion 98 at an end 93. In one embodiment, end portion 91 can be configured in a “U” shape. In other embodiments, end portion 80 can be configured in “L” or “V” shapes or be provided with no bends or curves, i.e., straight or substantially straight. First longitudinal side portion 94 and end portion 91 are provided such that a gap G exists between the two portions. The gap G is dimensioned to enable material of the wearer's show to fit therebetween.

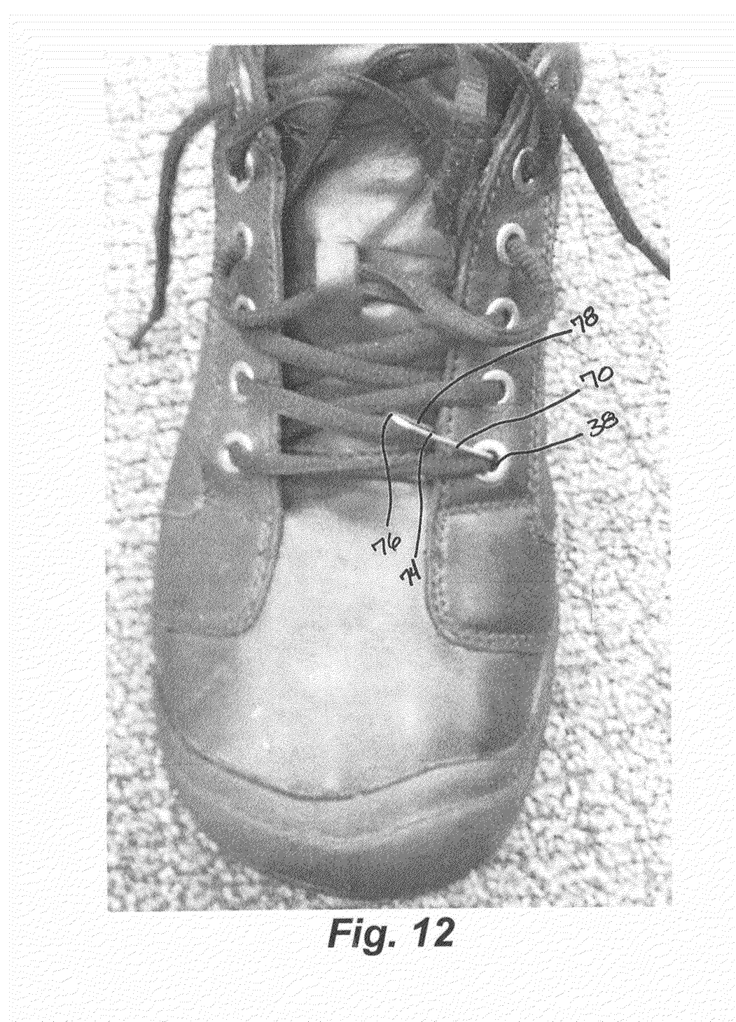

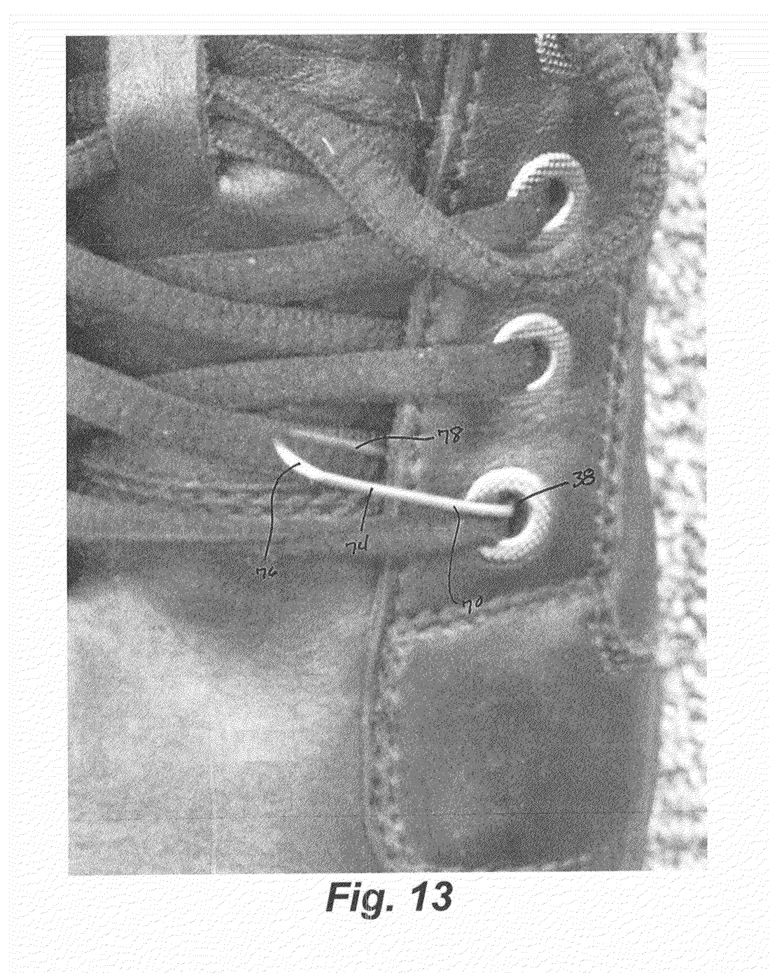

Referring to FIGS. 12-13, in operation, fastener clip 90 is threaded through eyelet 38, so that second longitudinal side portion 98, then curved end portion 96, and finally first longitudinal side portion 94 move through eyelet 38 until anchor portion 92 is in contact with eyelet 38. Hook fastener 26 can engage with curved end portion 96.

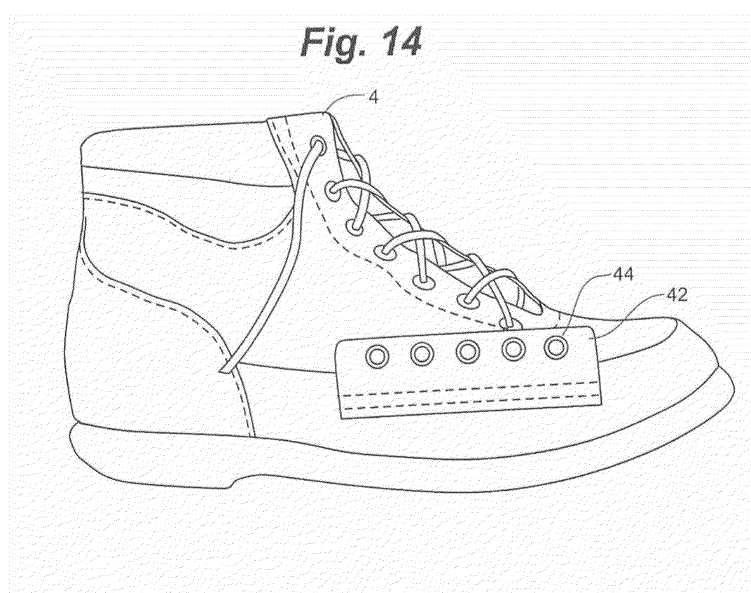

In yet another embodiment (as depicted in FIG. 14), at least one grommet assembly 42 comprising a plurality of grommets 44 releasably engageable by the hook fasteners 26 can be affixed to the shoe 4. Grommet assembly 42 can be sewn, riveted or adhered with adhesive to the user's shoe or other conventional means of affixing the grommet assembly 42. According to an embodiment of the present invention, the grommet assembly 42 can comprise a fabric or leather construction to flex with the user's shoe 4 during walking or running.

Wearers can customize the arrangement of the elastic strap 24 to accurately direct the tension force applied by the elastic strap 24 and maximize the effectiveness of the ankle foot orthotic 2. Foot drop can affect the various foot muscles differently from wearer to wearer and even from foot to foot, which can cause each afflicted foot to fall uniquely. For example, an afflicted foot can fall downwardly, roll or some combination thereof. A wearer can mix and match which ring anchor 40, fastener clip 90, or grommet 44 to engage with the hook fasteners 26. For example, a wearer can engage both hook fasteners 26 to the same ring anchor 40, fastener clip 90, or grommet 44 to maximize the force applied to the same part of the shoe 4. Similarly, the hook fasteners 26 can be positioned such that the elastic strap 24 crosses over the top of the shoe 4 or is uncrossed to properly direct the tension force.

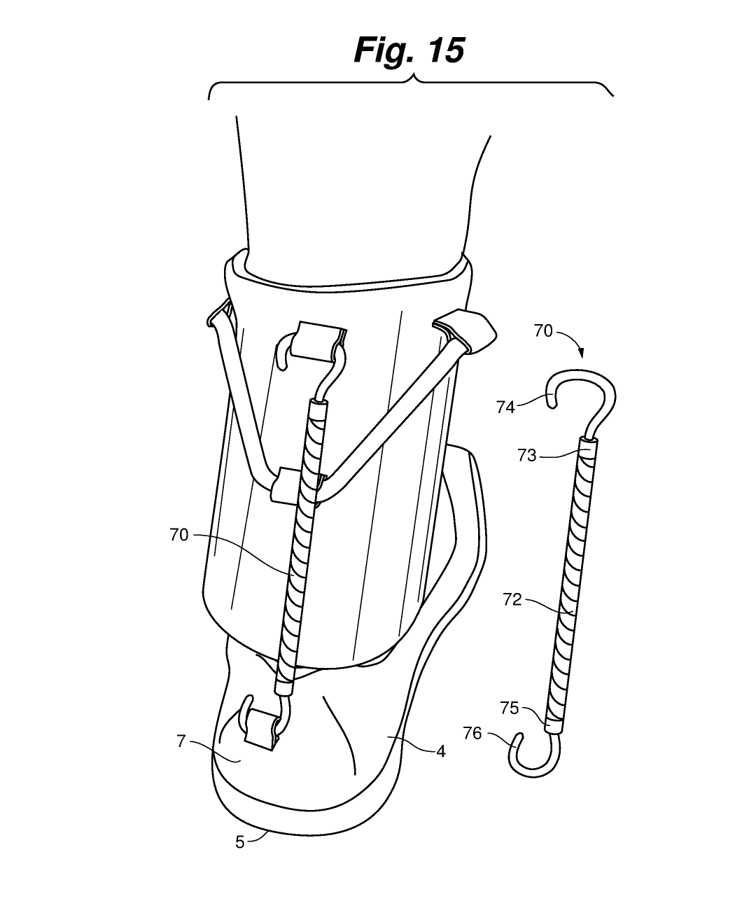

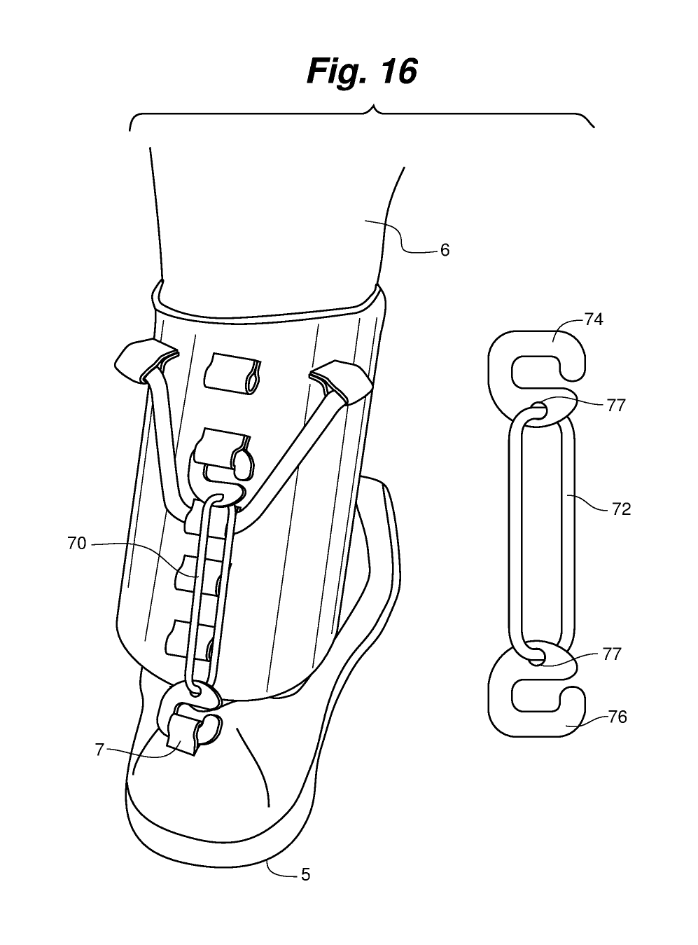

Referring to FIGS. 15-17, in one embodiment, ankle-foot orthotic 2 can further comprise a heel holder 70 to keep the heel 5 of the wearer's shoe 4 snug against the wearer's foot. In one embodiment, heel holder 70 can be comprised of an elastic strap 72 have a first hook 74 and a second hook 76. For example, as depicted in FIG. 15, elastic strap 72 can have a first end 73 fixedly coupled to first hook 74 and second end 75 fixedly coupled to second hook 76. Alternatively, as depicted in FIG. 16, elastic strap 72 can be a loop, threadedly coupled to one or more apertures 77 on first hook 74 and second hook 76.

In operation, first hook 74 can be coupled to strap loop 22, while second hook 76 can be coupled to an aft portion 7 of the wearer's shoe 4. The elasticity of elastic strap 72 can be selected to counteract the tension of elastic strap 24 connected to the fore portion of the wearer's shoe, thereby keeping the heel 5 of the wearer's shoe 4 snug against the wearer's foot.

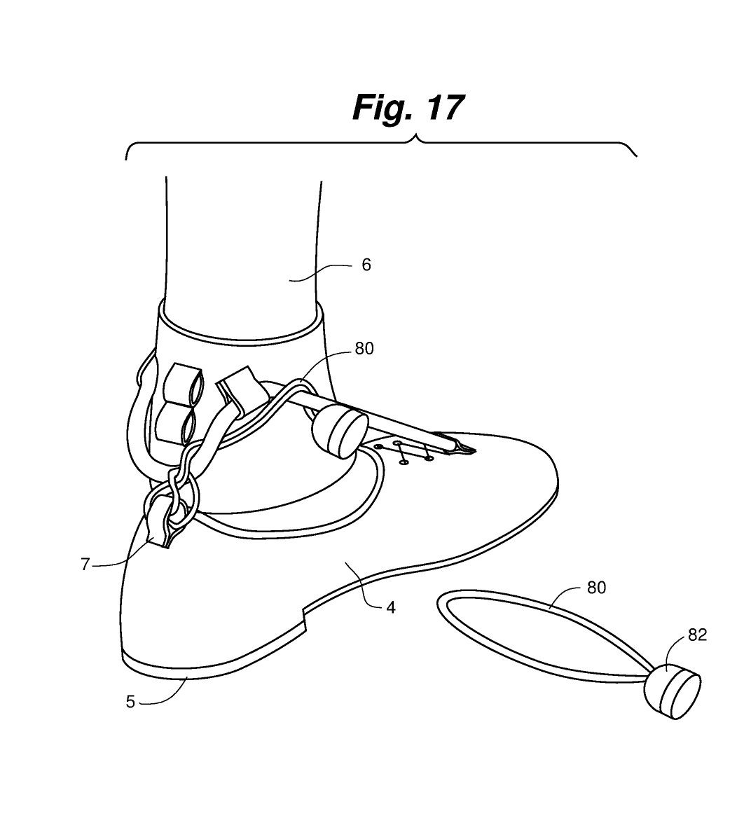

In another embodiment, as depicted in FIG. 17, heel holder 70 can comprise an elastic loop 80 threadedly coupled to an aft portion 7 of the wearer's shoe 4. In one embodiment, a grip 82 can be coupled to one portion of elastic loop 80 to provide an easy gripping point for heel holder 70. In operation, a wearer can insert their foot through elastic loop 80, so that the tension created by elastic loop 80 counteracts the tension of elastic strap 24 connected to the fore portion of the wearer's shoe, thereby keeping the heel 5 of the wearer's shoe 4 snug against the wearer's foot. In one embodiment, elastic strap 24 can be threaded through elastic loop 80, thereby retaining elastic loop 80 in desirable position on the wearer's ankle.

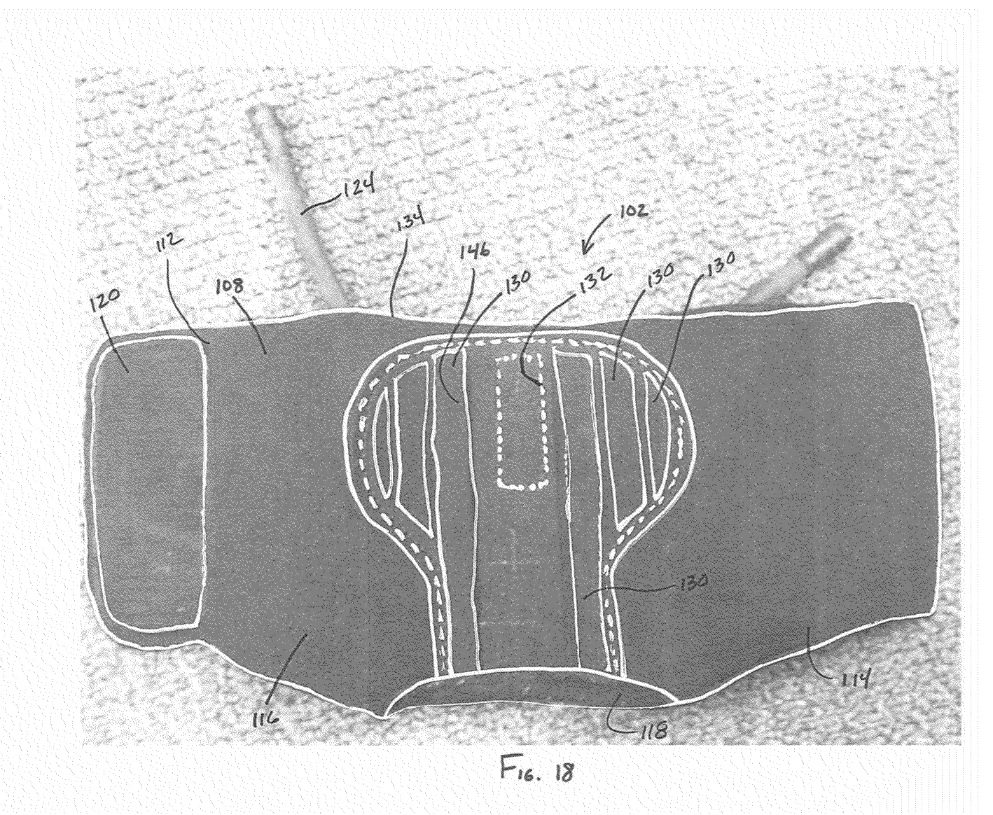

Referring to FIG. 18, another ankle-foot orthotic 102 according to an embodiment of the present invention is depicted. In one embodiment, ankle-foot orthotic 102 generally comprises an ankle brace 108 and an elastic strap 124. In one embodiment, ankle brace 108 can comprise a first wing 112 and a second wing 114 and defines an interior face 116 and an exterior face 118. Ankle-foot orthotic 102 operates similarly to ankle-foot orthotic 2 (as described in relation to FIGS. 1-6) in that ankle brace fastener 120 releasably engages the first wing 112 to the second wing 114 so that ankle brace 108 forms a generally cylindrical shape around a wearer's ankle and is shaped to conform around the ankle. Ankle brace fastener 120 can comprise, for example, VELCRO®, a hook and loop fastener, an elastic fastener, a button fastener or any other temporary fastening means.

In one embodiment, ankle brace 108 is shaped to conform to the ankle area and comprises built-in support 146. Built-in support 146 can be fixedly sandwiched between interior face 116 and exterior face 118 and can be comprised of a plurality of longitudinal support members 130 of varying lengths and configurations. In an embodiment, longitudinal support members 130 can generally define rectangles, quadrilaterals, circular segments, or any combination thereof. Longitudinal support members 130 can be comprised of a stiff yet flexible material, for example, a plastic or metal, providing structure to the built-in support 146 while allowing for movement.

In one embodiment, a plurality of longitudinal support members 130 can be provided. The plurality of longitudinal support members 130 can be of different lengths, and can be positioned substantially parallel to each other, separated by a distance from an adjacent longitudinal support member 130. The horizontal width of and distance between longitudinal support members 130 can be sufficient to enable the longitudinal support members 130 to conform around the wearer's ankle area when the ankle-foot orthotic 102 is placed around the ankle. The vertical length of each longitudinal support member 130 can be chosen so that when the ankle-foot orthotic 102 is placed around the ankle, the longitudinal support members 130 do not make contact with the lateral malleolus area of the fibula and medial malleolus area of the tibia. A benefit of this is that while the ankle-foot orthotic 102 can be form fitting and provide sufficient structure and support, it still enables mobility of the ankle and reduces pressure points.

In one embodiment, strap loop 22 is fixedly attached to the built-in support 102, for example, by stitching strap loop 22 to the exterior face 118 and the interior face 116 with thread 132. In one embodiment, strap loop 22 is positioned on exterior face 118 and is fixedly attached between two of the longitudinal support members 130 so that strap loop 22 is positioned near the top 134 of the ankle brace 108. Elastic strap 124 is threadable through strap loop 22. In another embodiment, the strap loop 22 is fixedly attached to exterior face 118 only.

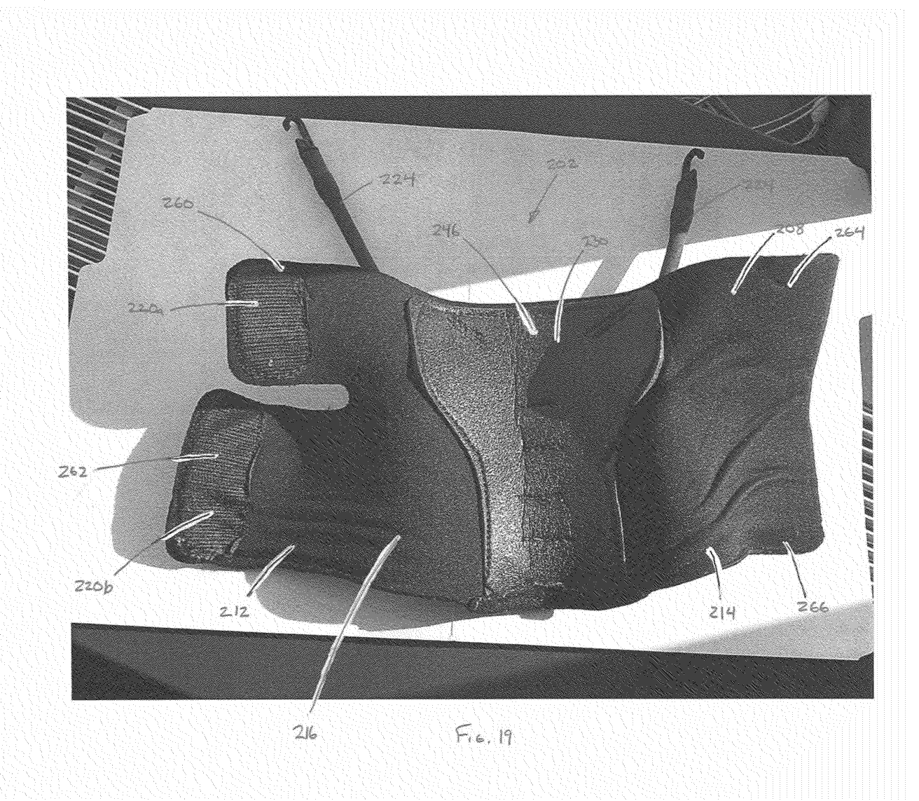

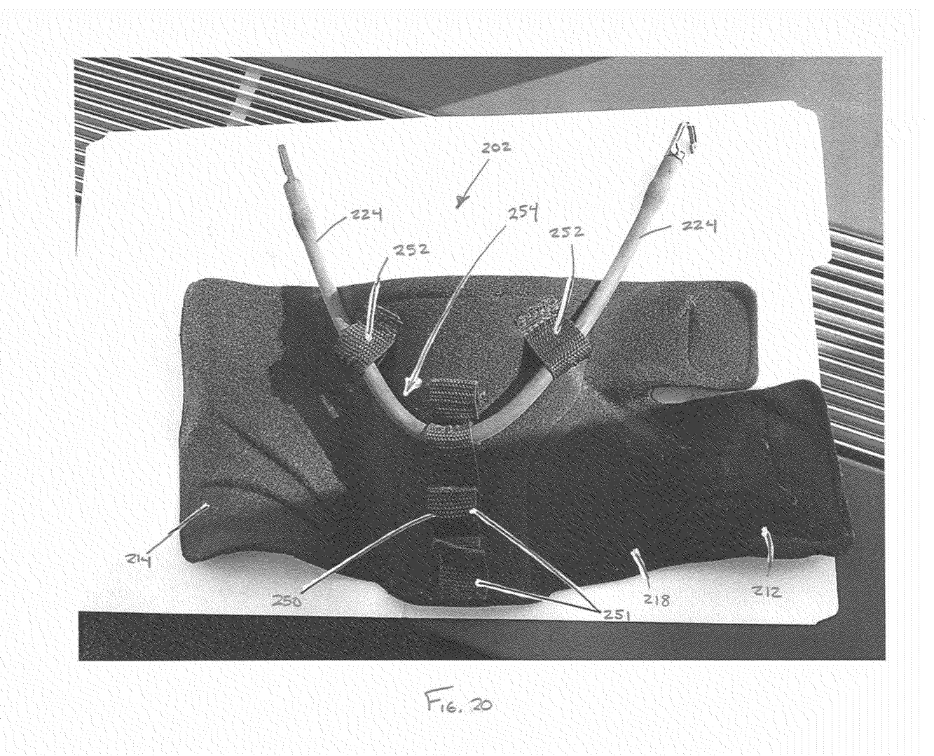

Referring to FIGS. 19-20, another ankle-foot orthotic 202 according to an embodiment of the present invention is depicted. In one embodiment, ankle-foot orthotic 202 generally comprises an ankle brace 208 and an elastic strap 224. In one embodiment, ankle brace 208 can comprise a first wing 212 and a second wing 214 and defines an interior face 216 and an exterior face 218. In one embodiment, first wing 212 can be split into an upper tab 260 and a lower tab 262. Upper tab 260 and lower tab 262 can include ankle brace fasteners 220a-b. Ankle brace fasteners 220a-b can comprise, for example, VELCRO®, a hook and loop fastener, an elastic fastener, a button fastener or any other temporary fastening means.

In one embodiment, ankle brace 208 is shaped to conform to the ankle area and comprises built-in support 246, which can be integrated into ankle brace 208. Built-in support 246 can be comprised of a unitary support member 230 with the unitary support member 230 being manufactured of a stiff yet flexible material that is able to wrap partially around the ankle area while still providing sufficient support. The unitary support member 230 can be molded or shaped so that when in operation, unitary support member 230 does not make contact with the lateral malleolus area of the fibula and medial malleolus area of the tibia.

In one embodiment, ankle brace 208 can comprise tension adjustment assembly 254 for adjusting the effective length of the elastic strap 224. The effective length of the elastic strap 224 is the portion of the elastic strap 224 extending from the rear of the wearer's ankle 6 to the wearer's shoe 4. Tension adjustment assembly 254 comprises a multi-loop primary strap loop 250 and two secondary strap loops 252 for receiving the elastic strap 224. Primary strap loop 250 comprises a plurality of sub-loops 251. In one embodiment, primary strap loop 250 and the secondary strap loops 252 are affixed to the exterior face 218 of the ankle brace 208. In operation, primary strap loop 250 and secondary strap loops 252 bend the elastic strap 224 into a U-shape within the tension adjustment assembly 254. Threading the elastic strap 224 through the various subloops 251 of the primary strap loop 250 changes the size of the U-shaped portion of the elastic strap 224, thereby changing the effective length of the elastic strap 224. Changing the effective length of the elastic strap 224 changes the tension force applied by the elastic strap 224.

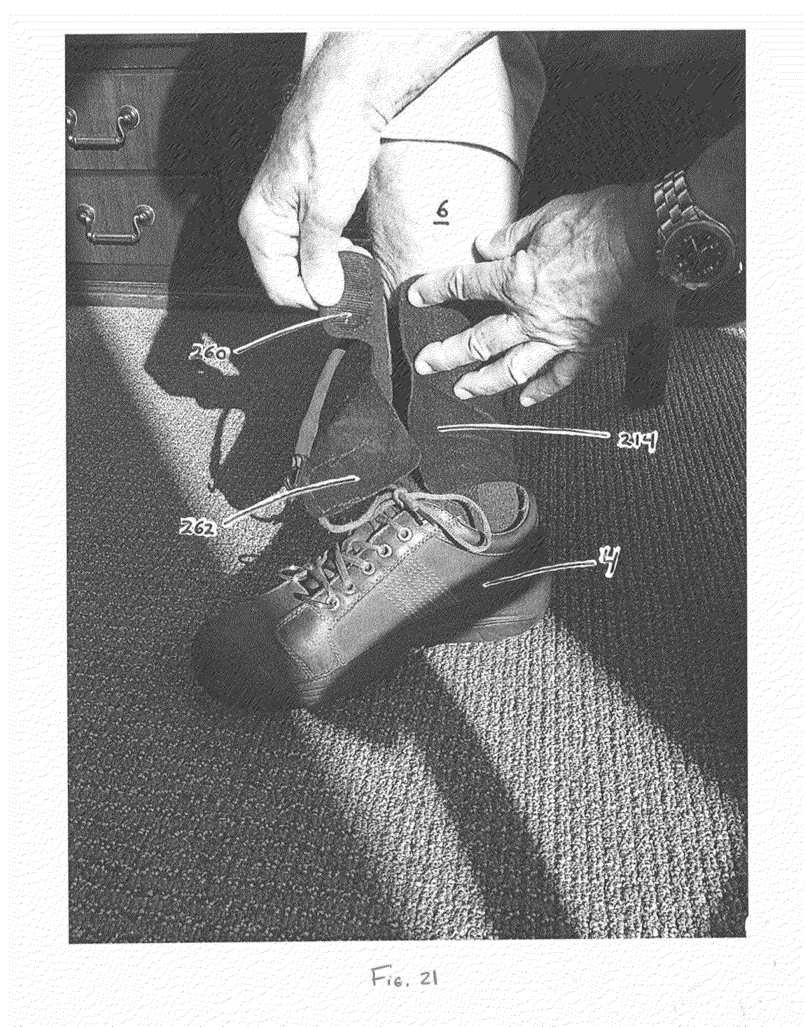

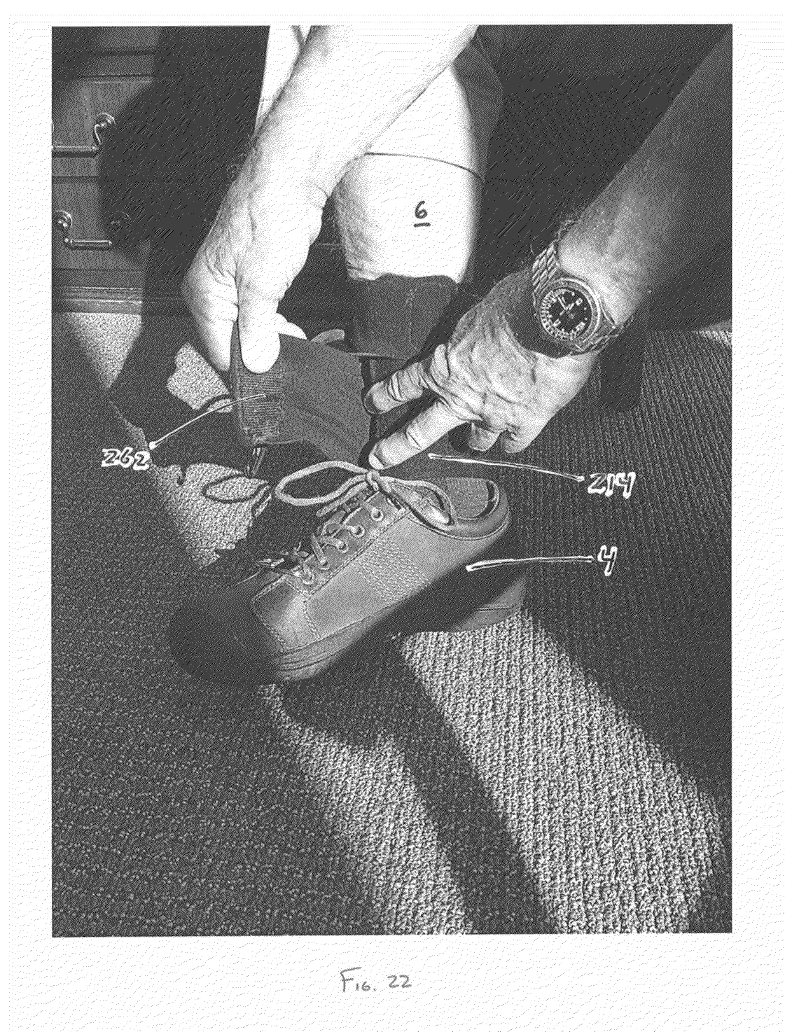

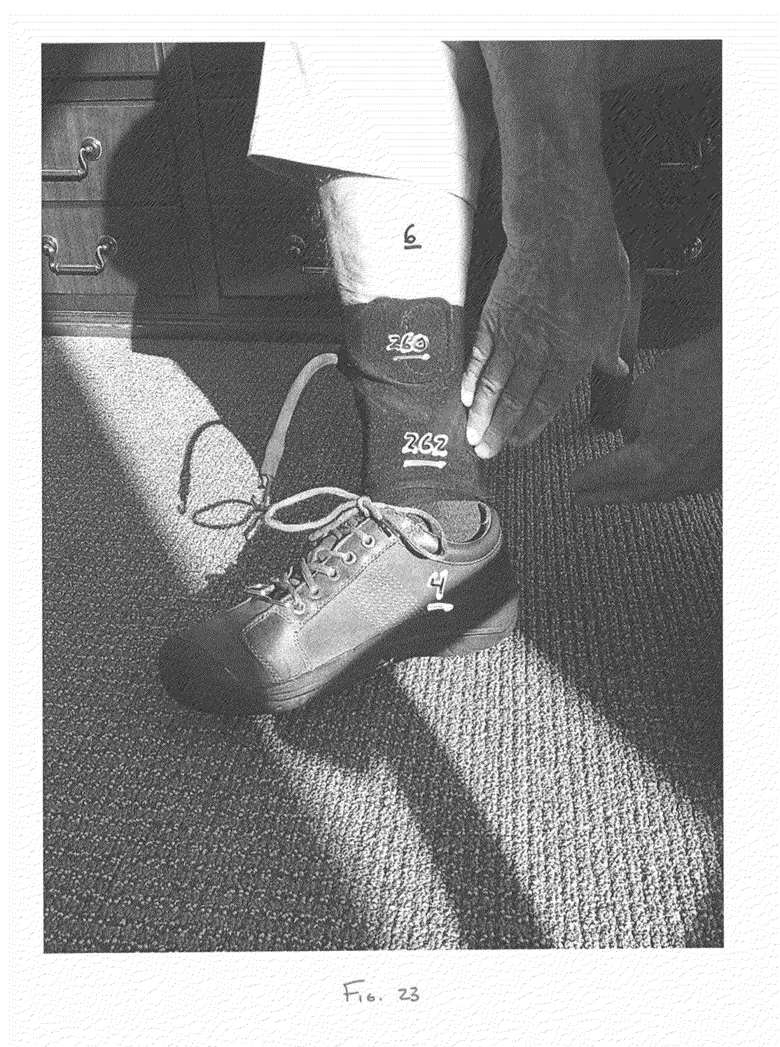

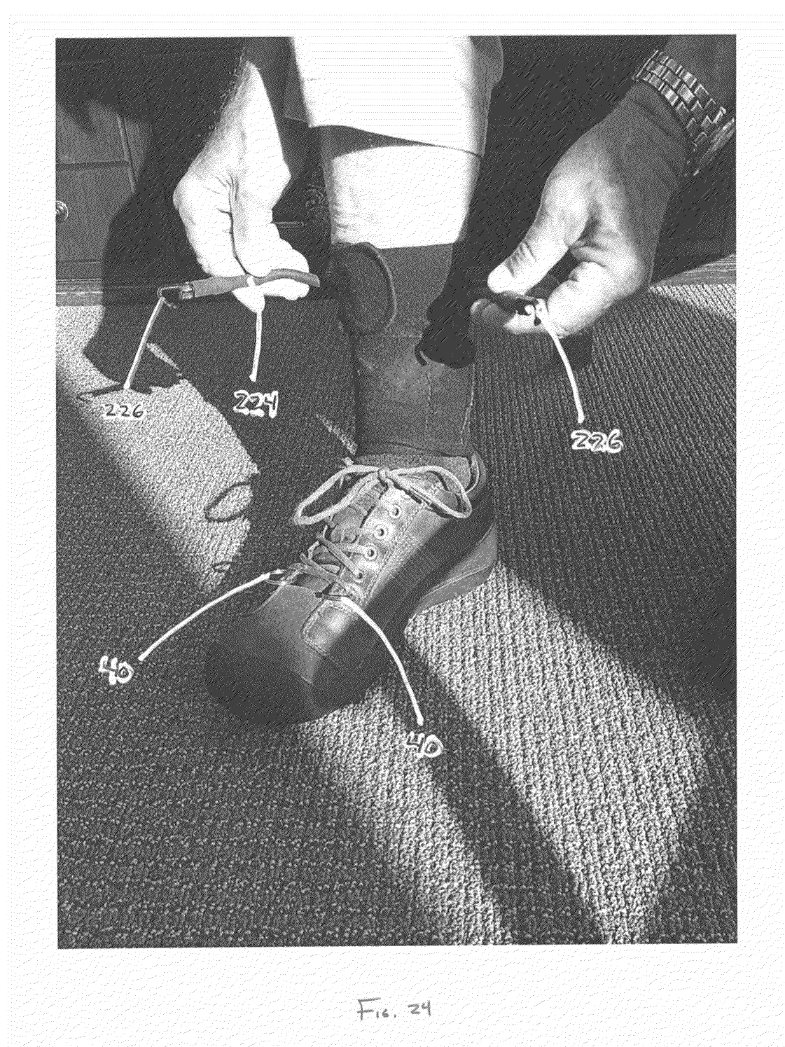

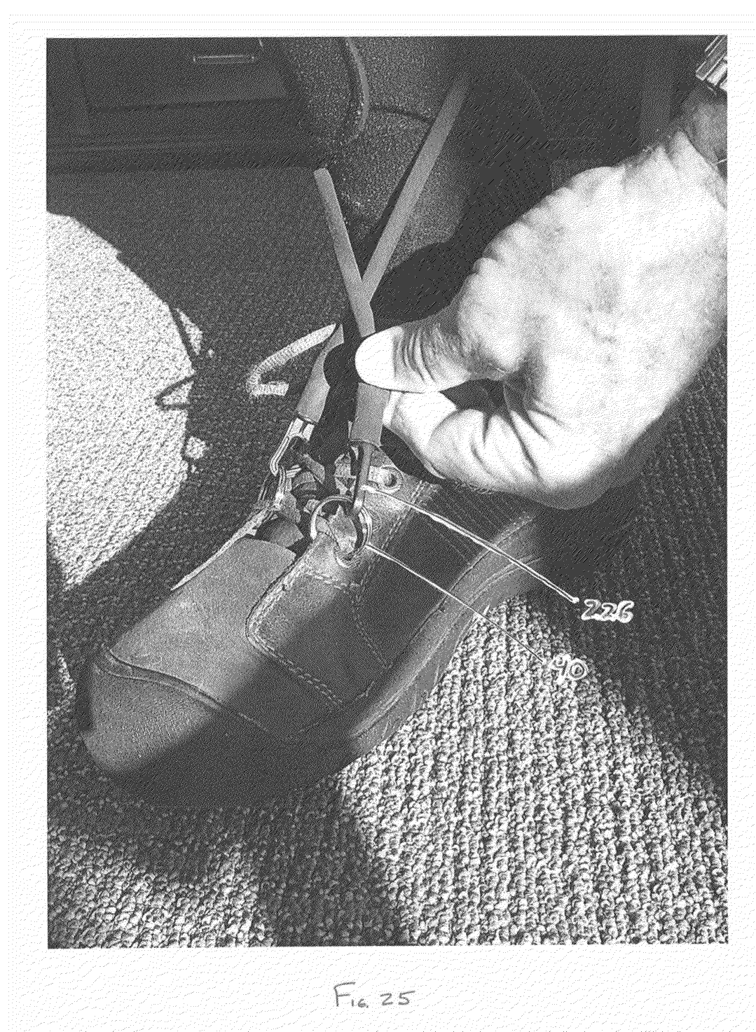

Referring to FIGS. 21-25, in operation, the first and second wings 212, 214 of the ankle brace 208 are wrapped around the wearer's ankle 6 such that the multi-loop primary strap loop 250 extends outwardly from the rear of the wearer's ankle 6. In this configuration, elastic strap 224 is threading through one or more of the subloops 251 of the primary strap loop 250 and secondary strap loops 252, as it wraps around the ankle brace 208 and the wearer's ankle 6 before engaging the wearer's shoes 4. Wrapping the elastic strap 224 around the wearer's ankle 6 and bending the elastic strap 224 into a U-shape within the tension adjustment assembly 254 ensures the correct tension is applied along the elastic strap 224 and properly angles the elastic strap 224 to maintain the wearer's shoe 4 in the proper position.

Ankle-foot orthotic 202 operates similarly to ankle-foot orthotic 8 (as described with references to FIGS. 1-9) in that ankle brace fastener 220 releasably engages the first wing 212 to the second wing 214 so that ankle brace 208 forms a generally cylindrical shape around a wearer's ankle and is shaped to conform around the ankle. Upper tab 260 and lower tab 262 present the additional advantage of being able to tailor the fit of ankle-foot orthotic 202 to a wearer's ankle 6. For example, upper tab 260 can be wrapped tighter around a wearer's ankle 6 than lower tab 262, so that the top portion 264 of ankle-foot orthotic 202 fits more snuggly around a wearer's ankle 6 than lower portion 266. Conversely, a wearer may prefer to have the lower portion 266 fit more snuggly. Upper and lower tabs 260, 262 can also be connected to second wing 214, such that the angle between upper and lower tabs 260, 262 varies, thereby causing one portion of the ankle-foot orthotic 202 to fit more snuggly than other portions and to enable the wearer to customize the fit of ankle-foot orthotic 202 and to reduce pressure points.

Various embodiments of the present disclosure have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of embodiments.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

1. A customizable ankle-foot orthotic for treating foot drop that that enables a wearer to adjust the fit of the ankle-foot orthotic against the wearer's ankle for the purpose of reducing pressure points while ensuring adequate support, the ankle-foot orthotic comprising:
an ankle brace adapted for installation around a wearer's ankle, the ankle brace including an exterior face and an interior face, the interior face positionable against the wearer's ankle, the ankle brace further including a first wing and a second wing, the first wing defining a first tab and a second tab, wherein the first and second tabs are independently configurable to overlap and selectively couple to a portion of the second wing to enable the wearer to customize the fit of the interior face against the wearer's ankle;
a tension adjustment assembly positioned on the exterior face of the ankle brace so as to protrude outwardly from the rear of the ankle brace when the ankle brace is wrapped around the wearer's ankle, the tension adjustment assembly including one or more strap loops; and
an elastic strap operably linking the ankle brace to a wearer's shoe for applying an upward resistive force to the wearer's shoe to prevent inadvertent downward movement of a wearer's foot, wherein the elastic strap is threadable through the one or more strap loops such that the elastic strap wraps around the exterior face of the ankle brace and extends at a downward angle to engage the wearer's shoe.

2. The ankle-foot orthotic of claim 1, further comprising a built-in support integrated into the ankle brace and comprising a saddle-shape for contouring to the wearer's ankle.

3. The ankle-foot orthotic of claim 1, wherein the first and second tabs of the first wing each comprise an adhering surface adapted to releasably adhere to the second wing.

4. The ankle-foot orthotic of claim 1, wherein the elastic strap defines a first end and a second end, each further comprising at least one hook fastener for releasably engaging any looped structures on the shoe.

5. The ankle-foot orthotic of claim 1, wherein the tension adjustment assembly comprises a primary strap loop having a plurality of sub-loops.

6. The ankle-foot orthotic of claim 5, wherein the tension adjustment assembly further comprises:
a first secondary strap loop for receiving the elastic strap; and
a second secondary strap loop for receiving the elastic strap;
wherein a portion of the elastic strap is bendable into a u-shape by threading the elastic strap through one of the sub-loops of the primary strap loop and the secondary strap loops, wherein changing the size of the u-shaped portion changes the upward resistive applied by the elastic strap.

7. The ankle-foot orthotic of claim 6, wherein threading the elastic strap through different sub-loops of the primary strap loop changes the size of the u-shaped portion of the elastic strap.

8. The ankle-foot orthotic of claim 1, wherein the elastic strap includes a first hook fastener and a second hook fastener positioned at opposite ends of the elastic strap.

9. The ankle-foot orthotic of claim 8, further comprising one or more engagement mechanisms positionable on the wearer's shoe configured to couple with at least one of the hook fasteners.

10. The ankle-foot orthotic of claim 9, wherein one of the one or more engagement mechanisms is a ring anchor.
